(19) United States
(12) Patent Application Publication
(10) Pub. No.: US 2003/0176810 A1
(43) Pub. Date: Sep. 18, 2003

(54) Thermography catheter

(76) Inventors: Tracy D. Maahs, Rancho Santa Margarita, CA (US); Jesus Flores, Perris, CA (US); Thomas H. Campbell, Brentwood, CA (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/099,409

(22) Filed: Mar. 15, 2002

(51) Int. Cl. A61B 5/00

(52) U.S. Cl. 600/549

(57) ABSTRACT

An improved catheter capable of detecting metabolic activity within vascular anomalies is disclosed. The apparatus of the present invention includes a catheter having a distal end and a proximal end, an expandable body located at the distal end of the catheter, the expandable body comprising one or more expandable body arms, and at least one sensor integrally formed on each of the expandable body arms. The at least one sensor may include a variety of sensors, including ultrasonic sensors, flow sensors, thermal sensors, thermocouples, blood temperature sensors, electrical contact sensors, conductivity sensors, and infrared sensors.

### FIELD OF THE INVENTION

The present invention relates, generally, to thermography catheters and, more particularly, to thermography catheters which use an expandable slotted body, or “basket”, at their distal end to detect hot spots (areas with high metabolic activity) of the atherosclerotic plaque, vascular lesions, and aneurysms in human vessels.

### BACKGROUND OF THE INVENTION

Cardiovascular disease is one of the leading causes of death worldwide. For example, some recent studies have suggested that the rupture of plaque within a blood vessel may trigger 60% to 70% of fatal myocardial infarctions. Moreover, plaque erosion or ulceration may trigger 25% to 30% of fatal infarctions. The ability to locate and detect vulnerable plaque within a blood vessel may permit the treatment of the area prior to the ulceration or erosion of the plaque deposit. However, vulnerable plaques are often undetectable using conventional techniques such as angiography. Indeed, the majority of vulnerable plaques that led to an infarction occurred in coronary arteries that appeared normal or only mildly stenotic on angiograms performed prior to the infarction.

Studies of the composition of vulnerable plaque suggest that the presence of inflammatory cells, particularly inflammatory cells having a large lipid core, may be a powerful predictor of ulceration and/or imminent plaque rupture. For example, in plaque erosion the endothelium beneath a thrombus formed on the interior wall of a blood vessel may be replaced by or interspersed with inflammatory cells. Further, recent literature suggests that the presence of inflammatory cells within vulnerable plaque may be identified by detecting thermal discontinuities associated with the metabolic activity of these inflammatory cells, thereby permitting the identification and detection of vulnerable plaque. Moreover, it is generally known that activated inflammatory cells have a heat signature that differs from that of connective tissue cells.

In light of the foregoing, it is believed that one way of detecting whether specific plaque is vulnerable to rupture and/or ulceration involves measuring the temperature of the walls of arteries in the region of a deposit of plaque. Once vulnerable plaque is identified, localized treatments may be developed to specifically address the problems. These treatments may include, for example, the localized delivery of therapeutic drugs to the area or thermal therapy.

Several devices capable of examining the thermal characteristics of vulnerable plaque have been developed. For example, the commonly assigned U.S. Pat. No. 6,245,026, issued Jun. 12, 2001, which is incorporated by reference in its entirety herein, describes a number of thermography devices capable of being introduced into a blood vessel of a patient, advanced to a area of interest within the body of a patient, and capable of examining the thermal characteristics of plaque deposits. In addition, alternate thermography catheters are described in U.S. Pat. Nos. 5,871,449 to Brown, U.S. Pat. No. 5,935,075 to Casscells et al. and U.S. Pat. No. 5,924,997 to Campbell, each of which are incorporated herein by reference.

While these devices have been successful in identifying vulnerable plaque within a blood vessel, several potential improvements to these devices have been identified. For example, ideally during use the thermal sensors located on the catheter would be capable of contacting the endothelium of the blood vessel thereby providing accurate temperature readings of the tissue. However, it would be advantageous to provide a device having sensors that contact that vessel wall directly but also minimize contact between the catheter and the endothelium, so as to minimize any immediate or long-term effects from repeated or excessive contact with the catheter. Furthermore, it would be advantageous to provide a device having a lower profile than those disclosed in the aforementioned patents.

In view of the foregoing, there is an ongoing need for an improved low profile, atraumatic thermography catheter which is capable of locating, identifying, and treating inflamed plaque and/or areas of thermal discontinuity within the blood vessels of a patient.

### SUMMARY OF THE INVENTION

To achieve the forgoing and other objects of the present invention, atraumatic interventional tools are described that are suitable for measuring the temperature of or temperature variations in a vessel wall in the body of a patient. Additionally, these interventional tools may include various integrated therapy embodiments or may serve as a delivery platform for a therapeutic working tool or other device.

Detecting heat associated with metabolically active inflammatory cells that are associated with vulnerable coronary plaques can be achieved with the use of small thermal sensors such as thermocouples. Thermocouples may consist of two wires made from dissimilar metals that are joined at the distal tip by welding or soldering. These thermal sensors have been used for years in electrophysiology catheters to monitor tissue temperature during ablation.

In one embodiment of the present invention, thermocouples run the length of the interventional device. These thermocouples are terminated in the proximal end of the interventional device so that the device can be attached to the monitoring equipment directly or through the use of an interconnect type cable. At the distal end of the interventional device the thermocouples fan out and are integrally attached to the individual struts of an expandable slotted body. The thermocouples are attached to the individual struts of the expandable slotted body with an adhesive such as epoxy or cyanoacrylate or by other mechanical means such as welding or soldering. In an alternate embodiment, the thermocouples may be integrally attached to expandable arms which expand individually outward from the device.

It will become evident to those skilled in the art that these thermocouples may be any one or a combination of any appropriate designs available to industry, or of customized origin, depending on the needs of a particular device.

Flex circuit technology, also known as “flexible printed wiring” or “flex print,” is already established as a way to create a multiplicity of parallel wires in a tiny space and is used in applications where compactness and flexibility are required. Flex circuit technology is currently used in the manufacture of hearing aids, ultrasonic probe heads, cardiac pacemakers and defibrillators. These same principles could also be used to make a thermocouple circuit.

In an alternate embodiment of the present invention, flexible circuits run the length of the interventional device. These circuits are terminated in the proximal end of the interventional device so that the device can be attached to the monitoring equipment directly, or through the use of an interconnect type cable. At the distal end of the interventional device the flexible circuits fan out and are fixedly attached to the individual struts of the expandable slotted body. Various construction techniques can be utilized to create thermal sensor circuits (thermal sensor circuit) that operate in a range from 20 to 80 ohms, based on the particular needs of a specific catheter.

The flexible circuits are attached to the individual struts of the expandable slotted body with an adhesive such as an epoxy or cyanoacrylate or by other mechanical means of treating the surface of the struts via plasma or chemical selective etching process.

By way of example, in one aspect of the present embodiment of the present invention, the thermal sensor circuits themselves are single sided flex circuits where a single conductor layer of either metal or conductive polymer is applied to a compliant dielectric film with sensor termination features accessible only from one side of the film.

In another aspect of the present embodiment the thermal sensor circuits themselves are multi-layer flex circuits having 3 or more layers of thermal sensor circuits which are interconnected by way of plated through-holes.

In still another aspect of the present embodiment the thermal sensor circuits are polymer thick film flex circuits that incorporate a specially formulated conductive or resistive ink that is screen printed onto the flexible substrate to create the thermal sensor circuit patterns.

It will become apparent to those skilled in the art that these conductive and or resistive inks can be any one of the many screenible types of ink that contain silver, carbon, or a silver/carbon mix to create the circuit patterns.

In yet another embodiment of the present invention, the intervention device incorporating thermal sensors allows for the introduction of a working element. In this embodiment, the working element may be an additional diagnostic tool such as an imaging wire or catheter, or pressure wire. In other embodiments, the working element may be of a therapeutic nature, depending on the needs of a particular device.

It will become apparent to those skilled in the art that introduction of the working element of the interventional tool could be accomplished by incorporating either “over-the-wire” or “rapid exchange” designs which are well known in the art of interventional cardiology catheters and construction in the flexible tubular member that supports communication between the distal and proximal ends.

Another embodiment of the present invention describes the means by which these thermal sensors display, collect, and store its data in a control box connected to the proximal end of the interventional device.

### BRIEF DESCRIPTION OF THE DRAWINGS

The apparatus of the present invention will be explained in more detail by way of the accompanying drawings, wherein:

FIG. 1 illustrates a longitudinal view of the distal section of the device in accordance with the embodiments described in the present disclosure;

FIG. 2 illustrates a longitudinal view of the distal section in a deployed state in accordance with the embodiments described in the present disclosure;

FIG. 3 illustrates a perspective view of the distal section in a deployed state in accordance with the embodiments described in the present disclosure;

FIG. 4 illustrates a perspective view of the sensor slot of the expandable slotted body of the present invention receiving a sensor device therein;

FIG. 5 illustrates a perspective view of the sensor device positioned within the sensor slot of the expandable slotted body of the present invention;

FIG. 6 illustrates a perspective view of the sensor device attached to the sensor slot of the expandable slotted body of the present invention;

FIG. 7 shows a cross-sectional view of the sensor device integrally positioned within the sensor slot of the expandable slotted body of the present invention;

FIG. 8 illustrates a cross sectional view of the distal section of the catheter taken at section 3-3 of FIG. 1 in accordance with the embodiments described in the present disclosure;

FIG. 9 illustrates a longitudinal perspective view of the distal section of the device in a deployed state in accordance with the embodiments described in the present disclosure;

FIG. 10 illustrates a longitudinal perspective view of the proximal handle section of the device in accordance with the embodiments described in the present disclosure; and

FIG. 11 illustrates a longitudinal perspective view of the catheter as a system in accordance with the embodiments described in the present disclosure.

### DETAILED DESCRIPTION OF THE DRAWINGS

Disclosed herein is a detailed description of various embodiments of the present invention. This description is not to be taken in a limiting sense, but is made merely for the purpose of illustrating the general principles of the invention. The overall organization of the detailed description is for the purpose of convenience only and is not intended to limit the scope of the present invention.

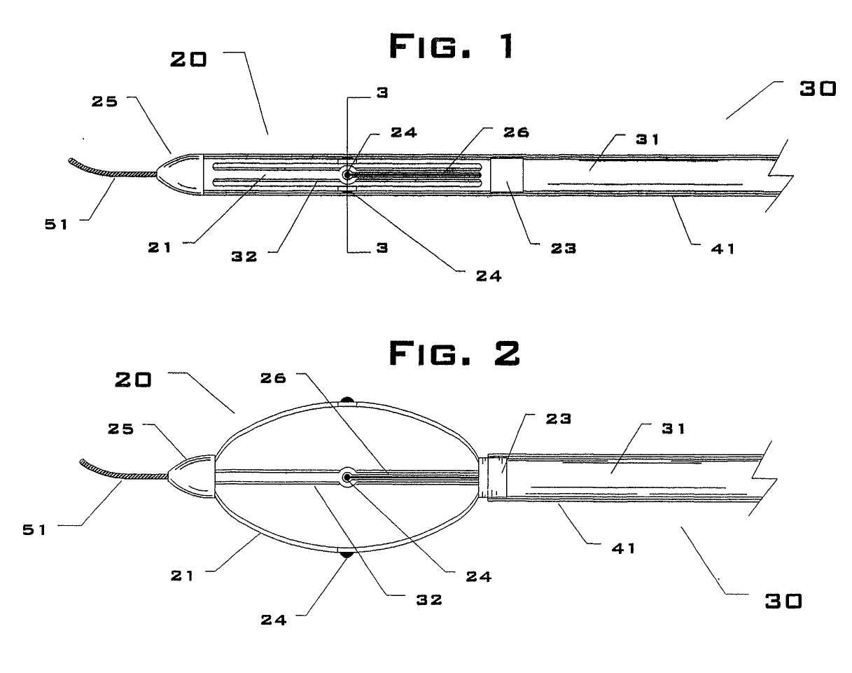

FIGS. 1, 2, and 4 show one embodiment of the present invention, wherein the distal section 20 of the basket catheter 90 comprises an expandable slotted body 21 and distal tip 25. The distal tip 25 serves as the exit point for the guide wire 51 which runs the length of the basket catheter 90 through the inner shaft 32, and as such, should be atraumatic in design and material so not to cause undo insult to the vessel wall while it is navigated to the desired investigative site. Insult to the endothelium could lead to immediate or long-term negative effects such as acute thrombosis, spasming, and or abrupt closure of the vessel all of which can trigger a heart attack.

In FIGS. 1 and 2 the distal tip 25 is conical shaped while in FIG. 4 it is elongate in shape. It will become apparent to those skilled in the art that any number of tip designs may be utilized in the distal section 20 depending on the needs of a particular basket catheter 90. The distal tip 25 may be manufactured from a plurality of materials including, for example, platinum, or a platinum iridium blend, and may be manufactured by micro-machining, or any one of the other commercially available processing methods such as powder metal molding (PMM). In an alternate embodiment, the atraumatic distal tip 25 of the present invention may be made from a radiopaque or echogenic material, thereby allowing the basket catheter 90 to be visualized by the physician in a variety of manners, including, for example, fluoroscopy, while navigating the device through the vasculature. This can easily be achieved by compounding one of many commercially available high molecular weight materials such as barium sulfate into the polymer used to fabricate the distal tip 25 of the basket catheter 90.

The distal tip 25 may be bonded to the distal end of the expandable slotted body 21 of the basket catheters 90 using a compatible adhesive, such as an epoxy or cyanoacrylate. For example, acceptable bonding may be achieved using Dymax® 205-CTH adhesive 27, a polyurethane oligomer mixture sold by Dymax Corporation of Torrington, Conn.

The expandable slotted body 21 is generally hollow in design and may be laser cut from tubing made from an expandable material or shape memory alloy (SMA) such as Nitinol®. The “as cut” diameter of the expandable tubing used to form the expandable slotted body 21 typically determines the overall diameter, or “crossing profile,” of the distal section 20 of the basket catheter 90. It will become apparent to those skilled in the art that the diameter of the expandable slotted body 21 can be processed from tubing or flat sheet to form expandable slotted bodies in a variety of diameters depending on the needs of a particular basket catheter 90. For example, the expandable slotted body may have a diameter from about 2 mm to about 5 mm. In an alternate embodiment, the expandable slotted body 21 may have a diameter as large as about 20 mm where the basket catheter 90 is used in the human aorta.

The expandable slotted body 21 may be shaped into its desired final deployed configuration and diameter by mounting the expandable slotted body 21 onto a shape setting mandrel, heating the expandable slotted body 21 above its transition temperature, and then cooling the assembly in a water bath.

The expandable slotted body 21 has one or more slotted body arms. In the illustrated embodiment of FIGS. 1 and 2 the expandable slotted body 21 is comprised of four separate slotted body arms 22. FIG. 4 illustrates an alternate embodiment of the expandable slotted body 21 having six separate slotted body arms 22. In one preferred embodiment, the expandable slotted body 21 has five separate slotted body arms 22. Those skilled in the art will appreciate that the number of slotted body arms may vary depending on the needs of a particular device.

Running down the midpoint of the slotted body arm 22 from the proximal end to the center point of the slotted body arm 22 is the sensor slot 26. The sensor slot 26 allows the sensor 24 to be integrally formed into a portion of the length of the slotted body arm 22 so that only the distal tip of the sensor 24 is exposed above the surface of the slotted body arm 22. This reduces the profile of slotted body arm 22.

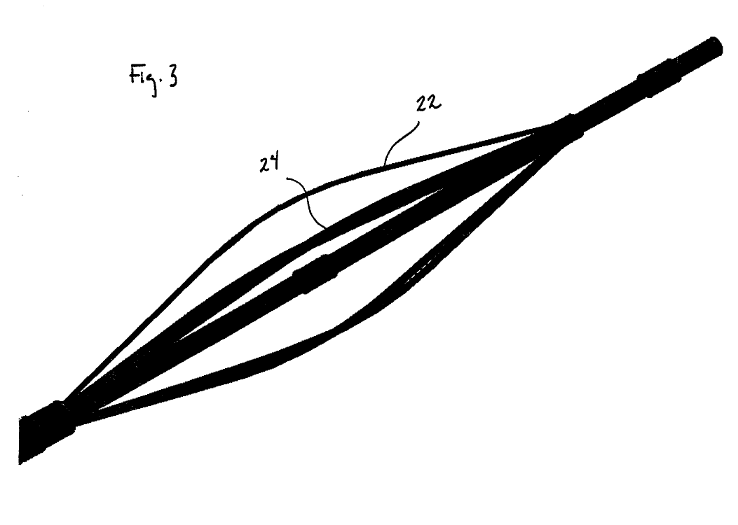

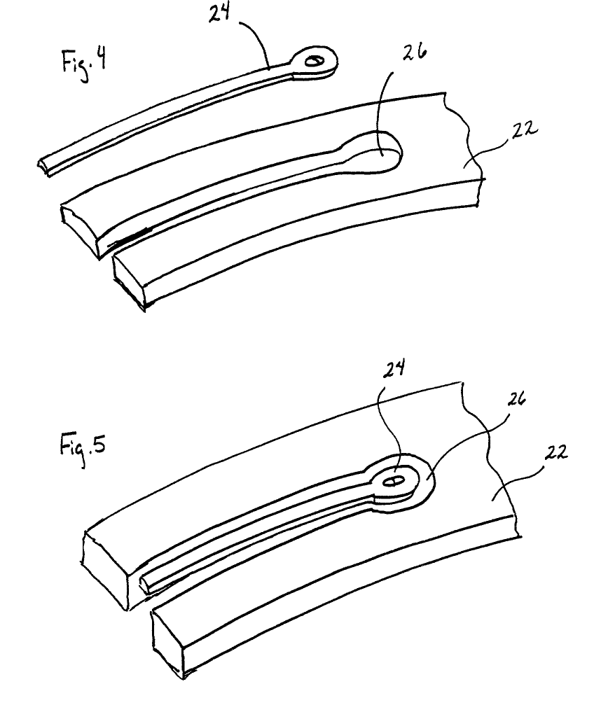

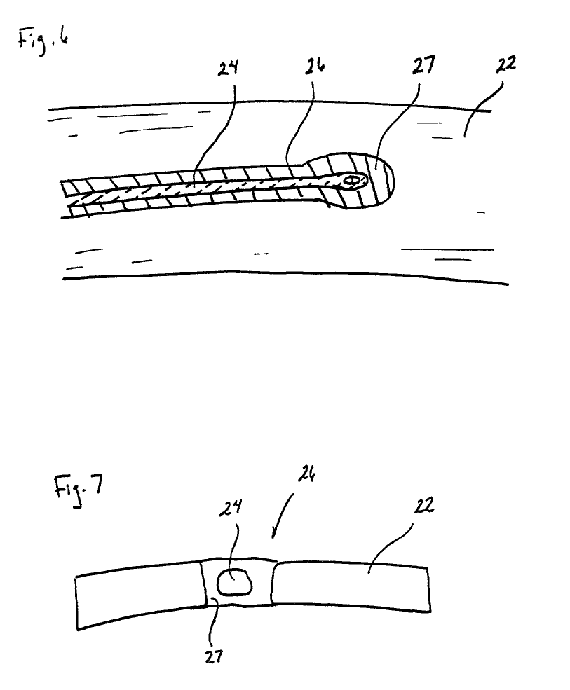

FIGS. 3-7 show various views of the slotted body arm 22 having a sensor slot 26 formed therein. As shown in FIGS. 4-7, the sensor slot 26 is longitudinally positioned along the slotted body arm 22 and is capable of contacting the internal vessel wall, thereby resulting in more accurate thermal measurements of tissue positioned proximate thereto. FIGS. 4-6 show perspective views of the slotted body arm 22 wherein a sensor slot 26 is receiving a thermocouple or other sensor device 24 therein. Thereafter, the thermocouple or other sensor device 24 may be adhesively attached to the slotted body arm 22 within the sensor slot 26 with an epoxy or other adhesive material 27. The sensor devices 24 communicate via one or more conductors 25 at least one external detection device (not shown), proximal to the device. Such external detection devices are well known to those of skill in the art and may include, for example, thermal detectors, monitors, computers, ohm meters, amp meters, voltage meters, and electromagnetic detectors.

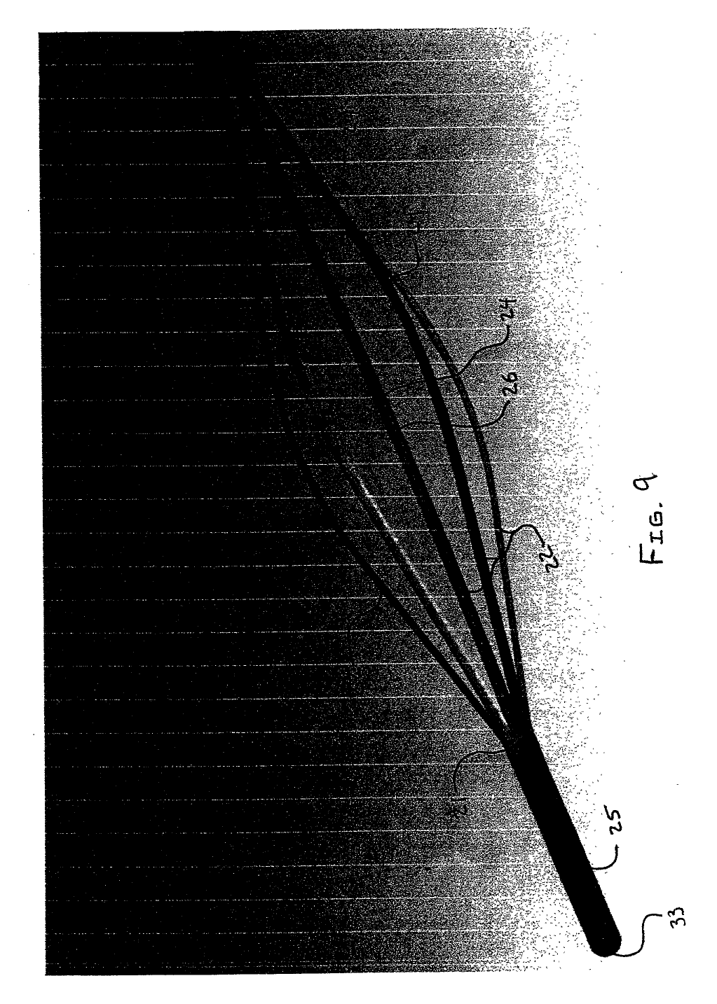

Those skilled in the art will appreciate that the instant invention provides for a reduction in the overall profile of the expandable slotted body 21 when compared to prior art devices, thereby permitting the present invention to be effectively used in smaller diameter locations within the body as compared with prior art systems. The sensor slot 26 is a slot that may be formed by laser etching or chemically etching the outer surface of the expandable tubing or sheet, prior to forming each of the individual slotted body arm's 22 that make up the final expandable slotted body 21. FIG. 7 shows a cross-sectional view of the sensor slot 26 having a thermocouple or other sensor device 24 positioned therein. Those skilled in the art will appreciate that the present invention may incorporate a variety of sensor devices, including, for example, ultrasonic sensors, flow sensors, thermal sensors, blood temperature sensors, electrical contact sensors, conductivity sensors, chemical sensors, and infrared sensors. As such, the present invention may be utilized to examine a number of characteristics of tissue within the body of a patient, including, without limitation, vessel wall temperature, blood temperature, fluorescence, luminescence, flow rate, and flow pressure.

As shown in FIG. 7, the thermocouple or other sensor device 24 may be adhesively attached to the sensor slot 26 utilizing a variety of materials known to those of skill in the art, such as 205-CTH epoxy 27. Each of the slotted body arm 22 includes a thermocouple 24 attached within its respective sensor slot 26. The thermocouple tip may be mounted to the center of the slotted body arm 22 so that when the expandable slotted body 21 of the basket catheter 90 is deployed in a vessel, the thermocouple tip is at the apex of the curve formed by the deployed SDA 22 and in contact with the vessel wall, thereby allowing the thermocouple 24 to record the temperature of the vessel surface. By contacting the vessel wall the thermocouple tip can provide a more accurate measurement of vessel temperature.

The epoxy 27 surrounds the distal section of the thermocouple and supports it in the sensor slot 26. The bonded thermocouples 24 run the proximal length of the basket catheter 90 through the central shaft 30 and continue through the handle assembly 40 and connector cable 57 where they terminate at the connector 62 mounted in the connector housing 61 of the proximal connector assembly 60. The proximal connector assembly 60 of the basket catheter 90 is connected to a monitor (not shown) that displays the temperature readings on a CRT screen for interpretation by the physician.

In an alternate embodiment, instead of an expandable slotted body, the device has one or more independently expandable, resilient body arms, each having its own sensor. An example of such a device is the vascular temperature measuring device disclosed in PCT International Publication Number WO 01/74263 A1, which is incorporated by reference herein. In this alternate embodiment of the present invention, the expandable or resilient body arms are also slotted body arms 22 having sensor slots 26 allowing the sensor 24 to be integrally formed into the length of the slotted body arm 22. As with other embodiments of the present invention, this alternate embodiment provides a device with a reduced profile and the ability to have the sensor more directly contact the vessel wall.

In an additional aspect of the present invention, the thermocouples 24 may be comprised of flexible circuits integrated into expandable body arms. A particular flexible circuit that is applicable to the present invention is disclosed in commonly assigned U.S. patent application Ser. No. 09/938963, to Rahdert et al, which is incorporated herein by reference.

In one embodiment of the flexible circuit thermocouple 24, the flexible circuit is comprised of polymer thick film flex circuit that incorporates a specially formulated conductive or resistive ink that is screen printed onto the flexible substrate to create the thermal sensor circuit patterns. This substrate is then adhered to the surface of each of the basket catheters 90 expandable body arm 22. In an alternate embodiment, the substrate can be adhered to independently expandable, resilient body arms which are not part of an expandable slotted body. As with all of the embodiments, the catheter can be provided with the appropriate number of body arms, such as four, five, six, or more.

It will become apparent to those skilled in the art that these conductive and or resistive inks can be any one of the many screenible types of ink that contain silver, carbon, or a silver/carbon mix to create the circuit patterns.

Additionally, FIGS. 1-2 and FIG. 9 show the location of the distal tip of the thermocouple 24 as being at the midpoint of each slotted body arm 22 shown on the expandable slotted body 21. It will be appreciated by those of ordinary skill in the art that the actual placement of the thermocouple 24 distal tip may be varied depending on the needs of a particular basket catheter 90, and that each slotted body arm 22 may include one or more thermocouples 24 along its overall length, especially when flexible circuit thermocouples 24 are utilized.

In FIGS. 1 and 2, the proximal end of the expandable slotted body 21 is attached to the distal end of the outer shaft 31 with the aid of a coupler 23. The coupler 23 may include generally tubular sleeves, wherein the expandable slotted body 21 and the outer shaft 31 maintain concentricity between the expandable slotted body 21 and the entire central shaft 30 of the basket catheter 90. It will become obvious to those skilled in the art that the coupler 23 may be manufactured from a polymer tube or a metal tube, and that the joint could be made using any number of various methods such as adhesive and or thermal bonding or force derived from dimpling or the use of interference fits.

With the expandable slotted body 21 shape set, deformation introduced below the transition temperature will be reversible. FIGS. 1 and 2 illustrate an embodiment of the present invention having an outer sheath 41 of the basket catheter 90. The outer sheath 41 is an elongate tubular member made from an extruded polymer which runs the length of the basket catheter 90 covering the central shaft 30 and expandable slotted body 21.

At its proximal end, the outer sheath 41 enters and continues through the strain relief 44 and terminates at the handle body 42 where it is adhesively attached to a barrel 46 (not shown) using any of a variety of adhesives. Exemplary adhesive include, for example, Loctite® 3321; a UV curable thixotropic adhesive, made by Loctite Corporation of Rocky Hill, Conn. The barrel may be appropriately sized to permit the back and forth movement along the inside of the handle body 42 through linear manipulation of the deployment knob 43.

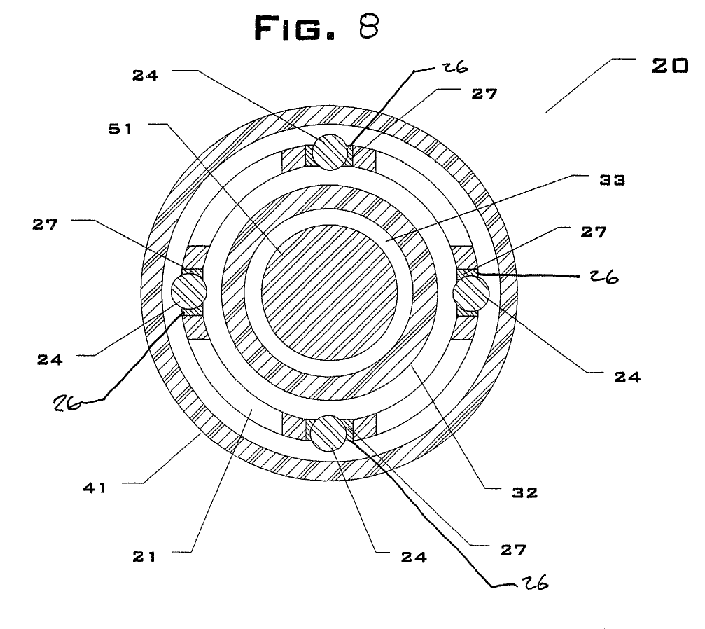

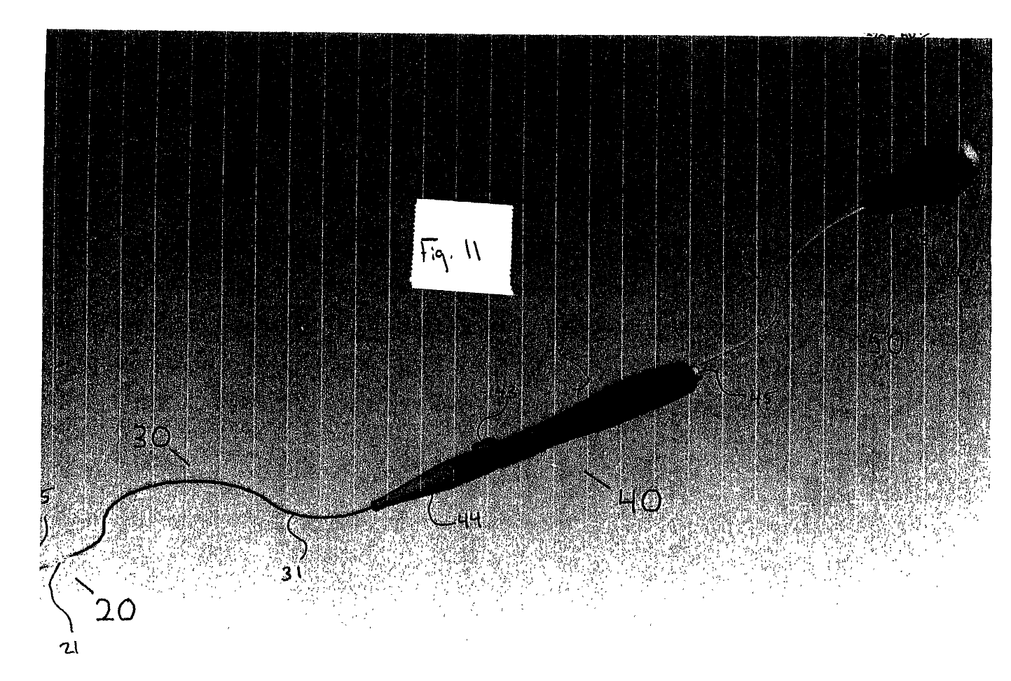

As shown in FIGS. 10 and 11, the deployment knob 43 may be attached to the barrel 46 in a variety of ways, such as, for example, by a dowel pin or spring pin. The handle slot 47 shown in FIG. 6 may allow for the proximal or distal travel of the barrel assembly. The handle slot 47 is positioned on the handle body 42 and serves as a mechanical stop. This feature provides the user with tactile information regarding the location of the distal tip of the outer sheath 41, in relationship to the distal or proximal end of the expandable slotted body 21.

By way of example, to deploy the expandable slotted body 21 in a target vessel, the user places his or her thumb on the top surface of the deployment knob 43 located on handle body 42. The user slides the deployment knob 43 in a proximal direction down the handle slot 47 which results in longitudinal movement of the outer sheath 41 in telescoping relation to the elongated body 32.

In FIG. 1 the outer sheath 41 is shown in a fully distal position where it makes contact with the proximal face of the distal tip 25. In this position, with the outer shaft 41 overlaying the expandable slotted body 21, the expandable slotted body 21 is manipulated into a low profile configuration that may be more easily navigated through a torturous location in the body such as a coronary artery.

It will be apparent to those skilled in the art that incorporating a trepan or spot-face feature onto the proximal face of the distal tip 25 will aid in preventing the distal tip of the outer sheath 41 from separating from the proximal end of the distal tip 25 and possibly snow-plowing the vascular wall as it traverses a tortuous bend in the anatomy, and will maintain the concentricity between the distal end of the outer sheath 41 and the distal tip 25.

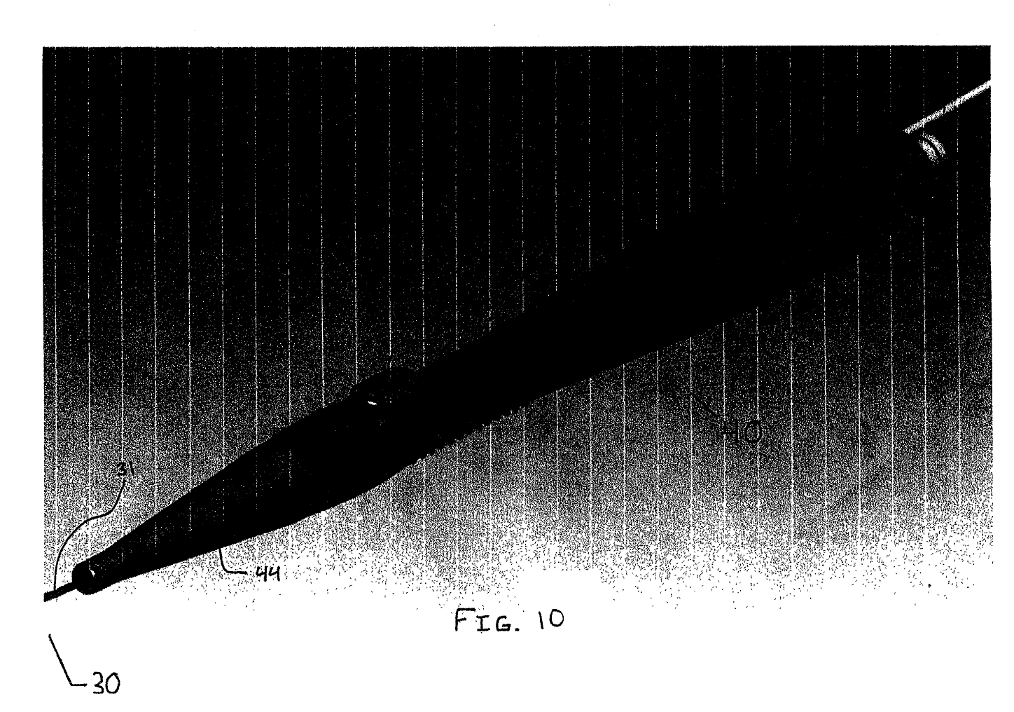

As shown in FIGS. 2 and 9, the outer sheath 41 has been brought proximally allowing the expandable slotted body 21 to deploy into its final pre-shaped diameter.

In FIG. 1 and FIG. 2 the distal section 20 of the basket catheter 90 is attached to the central shaft 30 with the aid of the coupler 23 and epoxy 27 as shown in FIG. 8. The central shaft 30 of the basket catheter 90 consists of an outer shaft 31 and an inner shaft 32, seen in the cross section FIG. 8. The outer shaft 31 is typically an extruded polymer tube that may or may not incorporate stainless steel braiding to enhance the torquability or the basket catheter 90.

The outer shaft 31 may incorporate a single lumen or a plurality of lumens depending on the needs of a particular basket catheter 90. A single central lumen may house the inner shaft 32 typically used as a conduit for the guide wire 51 and for flushing the central shaft 30 prior to introducing the basket catheter 90 into the body of a patient.

Separate lumens in the outer shaft 31 may be used to house the thermocouples 24 that run the length of the central shaft 30 separating and protecting them from the mechanism in the handle assembly 40 which is used to move the outer sheath 41 in a proximal or distal direction.

Additionally, an individual lumen of a multi-lumen outer shaft 31 may serve as a means of connecting the distal end of the basket catheter 90 to the handle assembly 40 with an SMA wire or stainless steel wire so that the proximal end of the wire, when attached to the barrel 46, can be manipulated with the deployment knob 43 allowing for steering of the distal tip 25 of the basket catheter 90.

It will be apparent to those skilled in the art that there will be numerous uses for the auxiliary lumens incorporated into the outer shaft 31 and whether they house steering wires or thermocouple wires the described embodiments are meant to be illustrative and not restrictive in nature.

FIG. 8 of the present invention shows the elongated body or inner shaft 32 of the of the central shaft 30. As previously mentioned, the inner shaft 32 typically serves as a conduit for the guide wire 50 which runs along the central lumen 33 of the inner shaft 32.

The inner shaft 32 runs the length of the basket catheter 90 its proximal end adhesively bonded into the flush port 45 at the proximal end of the handle assembly 40 the flush port 45 being adhesively bonded into the proximal end of the handle body 42.

The distal end of the inner shaft 32 exits the proximal end of the outer shaft 31 through the expandable slotted body 21 and into the proximal end of the distal tip 25 where it may be allowed to “float” in the proximal end of the distal tip 25 compensating for shortening of the distal section 20 that may occur when the expandable slotted body 21 is deployed in the target vessel.

Although exemplary embodiments of the present invention have been described in some detail herein, the present examples and embodiments are to be considered as illustrative and not restrictive. The invention is not to be limited to the details given, but may be modified freely within the scope of the appended claims, including equivalent constructions.

What is claimed:

1. An apparatus for sensing the interior of a blood vessel of a patient, comprising:
a catheter having a distal end and a proximal end;
an expandable body located at said distal end of said catheter; said expandable body comprising one or more expandable body arms; and
at least one sensor integrally formed on each of said one or more expandable body arms.

2. The apparatus of claim 1 wherein said proximal end of said catheter comprises a handle in communication with said distal end.

3. The apparatus of claim 2 further comprising:
a movable outer sheath attached to said handle, wherein said movable outer sheath is capable of moving from a distal position to a proximal position through the actuation of said handle; and
an elongated body in communication with said handle and said distal end, said elongated body contained within said outer sheath.

4. The apparatus of claim 3 wherein said elongated body includes at least one lumen formed therein.

5. The apparatus of claim 1 wherein said catheter is capable of engaging a guidewire.

6. The apparatus of claim 1 wherein said expandable body is capable of controllably expanding from a first non-deployed state to a second deployed state.

7. The apparatus of claim 6 wherein said expandable body is capable of contacting an interior wall of said blood vessel in said expanded state.

8. The apparatus of claim 1 wherein each of said one or more expandable body arms comprises a slotted body arm having at least one slot and said sensor is received within said slot.

9. The apparatus of claim 1 wherein said expandable body is manufactured from a shape memory alloy.

10. The apparatus of claim 9 wherein said shape memory alloy is Nitinol.

11. The apparatus of claim 1 wherein said catheter incorporates radio-opaque material therein.

12. The apparatus of claim 1 wherein said expandable body comprises four expandable body arms.

13. The apparatus of claim 1 wherein said expandable body comprises five expandable body arms

14. The apparatus of claim 1 wherein said at least one sensor is selected from the group consisting of ultrasonic sensors, flow sensors, thermal sensors, thermocouples, blood temperature sensors, electrical contact sensors, conductivity sensors, chemical sensors, and infrared sensors.

15. The apparatus of claim 1 wherein said at least one sensor further comprises a flex circuit material.

16. The apparatus of claim 1 further comprising:
one or more conductors in communication with said at least one sensor; and
at least one external detection device in communication with said one or more conductors.

17. The apparatus of claim 16 wherein said at least one external detection device includes at least one device selected from the group consisting of thermal detectors, video monitors, video displays, computers, ohm meters, amp meters, volt meters, and electromagnetic detectors.

18. An apparatus for sensing arterial plaque formed in a blood vessel of a patient, comprising:
a catheter having a distal end and a proximal end, said catheter capable of engaging a guidewire;
an expandable body located at said distal end of said catheter capable of controllably expanding from a first non-deployed state to a second deployed state, said expandable body comprising at least one slotted body arm having at least one slot; and
at least one sensor received within said slot of said slotted body arm.

19. The apparatus of claim 18 wherein said at least one sensor is selected from the group consisting of ultrasonic sensors, flow sensors, thermal sensors, thermocouples, blood temperature sensors, electrical contact sensors, conductivity sensors, chemical sensors, and infrared sensors.

20. The apparatus of claim 18 wherein said expandable body comprises four slotted body arms.

21. The apparatus of claim 18 wherein said expandable body comprises five slotted body arms

22. An apparatus for sensing arterial plaque formed in a blood vessel of a patient, comprising:
a catheter having a distal end and a proximal end, said catheter capable of engaging a guidewire;
an expandable body located at said distal end of said catheter capable of controllably expanding from a first non-deployed state to a second deployed state, said expandable body comprising at least one expandable body arm; and
at least one flexible circuit sensor integrally formed on said at least one expandable body arm.

23. The apparatus of claim 22 wherein said at least one sensor is selected from the group consisting of ultrasonic sensors, flow sensors, thermal sensors, thermocouples, blood temperature sensors, electrical contact sensors, conductivity sensors, chemical sensors, and infrared sensors.

24. The apparatus of claim 22 wherein said expandable body comprises four expandable body arms.

25. The apparatus of claim 22 wherein said expandable body comprises six expandable body arms.

26. An apparatus for examining the interior of a blood vessel of a patient, comprising:
a catheter having a distal end and a proximal end;
one or more independently expandable, resilient body arms located at said distal end of said catheter, each of said body arms having at least one slot; and
at least one sensor received within said slot of said one or more independently expandable, resilient body arms.

27. An apparatus for examining the interior of a blood vessel of a patient, comprising:
a catheter having a distal end and a proximal end;
one or more independently expandable, resilient body arms located at said distal end of said catheter; and
at least one flexible circuit sensor integrally formed on said at least one expandable body arm.
